(19) United States
(12) Patent Application Publication
(10) Pub. No.: US 2024/0374210 A1
(43) Pub. Date: Nov. 14, 2024

(54) LONG-TERM WEARABLE HEALTH MONITORING, DIAGNOSTICS AND THERAPY DEVICE

(71) Applicant: Nissim Igal LEVY, Savyon (IL)

(72) Inventor: Nissim Igal LEVY, Savyon (IL)

(21) Appl. No.: 18/287,458

(22) PCT Filed: Apr. 19, 2022
(86) PCT No.: PCT/IL2022/050406

§ 371 Date: Oct. 18, 2023

Related U.S. Application Data
(60) Provisional application No. 63/176,934, filed on Apr. 20, 2021.

(51) Int. Cl. A61B 5/00; A61B 5/0205; A61M 5/142; A61N 1/39

(52) U.S. Cl. CPC A61B 5/6804; A61B 5/02055; A61B 5/6803; A61B 5/6807; A61M 5/14244; A61N 1/3904

(57) ABSTRACT

A wearable system comprising: a body covering configured to cover a body part of a subject; at least one device attached to a surface of the body covering at a predetermined location with respect to the body part, the at least one device comprising: a skin-proximate layer configured to engage with a skin of the subject in a contact engagement at the predetermined location, an attachment layer configured to attach to the surface of the body covering, and a resilient layer disposed between the skin-proximate layer and the attachment layer, wherein the body covering and the resilient layer are configured to cooperate to exert a combined force which tends to urge the device towards the skin of the subject at the predetermined location.

### CROSS-REFERENCE TO RELATED APPLICATIONS

This application claims priority from U.S. Application Ser. No. 63/176,934, filed Apr. 20, 2021, the content of which is hereby incorporated in its entirety by reference.

### FIELD OF THE INVENTION

Embodiments of the present invention relate generally to devices and methods for wearable monitoring, diagnostics and therapeutic systems.

### BACKGROUND

In recent years, the use of medical sensing technology in combination with various communications platforms has provided new ways to monitor patients and communicate the results of the monitoring to their physician or caregiver. In some case, medical sensors are incorporated into articles of clothing, to monitor patients, e.g., for cardiovascular and other health-related problems, including respiratory problems.

Continuous long-term monitoring of patients provides more reliable and more accurate information on patient's status and may allow early and effective intervention, and medical assistance may be obtained based on monitored physiological characteristics before a particular health issue becomes fatal.

Continuous monitoring of patient's body movements enables patient's behavior analysis, correlating it with monitored data, and achieving more reliable and more accurate diagnostics overall.

However, currently available cardiovascular and other types of health monitoring and therapeutic systems may be inconvenient or impractical for extended use in monitoring and therapy.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in an embodiment, a wearable system comprising: a body covering configured to cover a body part of a subject; at least one device attached to a surface of the body covering at a predetermined location with respect to the body part, the at least one device comprising: a skin-proximate layer configured to engage with a skin of the subject in a contact engagement at the predetermined location, an attachment layer configured to attach to the surface of the body covering, and a resilient layer disposed between the skin-proximate layer and the attachment layer, wherein the body covering and the resilient layer are configured to cooperate to exert a combined force which tends to urge the device towards the skin of the subject at the predetermined location.

There is also provided, in an embodiment, method comprising: providing a wearable system comprising: a body covering configured to cover a body part of a subject; at least one device attached to a surface of the body covering at a predetermined location with respect to the body part, the at least one device comprising: a skin-proximate layer configured to engage with a skin of the subject in a contact engagement at the predetermined location, an attachment layer configured to attach to the surface of the body covering, and a resilient layer disposed between the skin-proximate layer and the attachment layer, wherein the body covering and the resilient layer are configured to cooperate to exert a combined force which tends to urge the device towards the skin of the subject at the predetermined location; and positioning the body covering about the body part so that the at least one device is located at the predetermined location.

In some embodiments, the skin-proximate layer comprises at least one sensor configured to produce a signal representative of a physiological characteristic of the subject.

In some embodiments, the at least one sensor is selected from the group consisting of: heart monitoring sensors, heart-rate monitoring sensors, electrocardiogram (ECG) sensors, body temperature sensors, breath-rate/respiration sensors, skin conductivity sensors, oxygen saturation sensors, blood perfusion sensors, blood pressure sensor, skin humidity sensors, electroencephalograph (EEG) sensors, and muscle activity sensors.

In some embodiments, the device comprises at least one therapy element configured to administer a therapy to the subject at the predetermined location.

In some embodiments, the method further comprises administering the therapy to the subject at the predetermined location.

In some embodiments, the therapy element is one of: an injection delivery device, a medication delivery device, a signal delivery device, and a defibrillator.

In some embodiments, the at least one therapy element comprises at least one canister configured to store a specified amount of a medication for application to the subject through an opening in the skin-proximate layer.

In some embodiments, the method further comprises applying the medication to the subject at the predetermined location

In some embodiments, the at least a portion of the body covering is formed with a predetermined amount of elasticity.

In some embodiments, the device comprises a wireless transceiver configured to receive and transmit data to a control module.

In some embodiments, the control module is an external control module, wherein the receiving and transmitting is carried out using wireless communication.

In some embodiments, the attachment layer is configured to attach to the surface of the body covering using one of: an adhesive, sewing, attachment through a hook and loop fastener, and buttons.

In some embodiments, the attachment layer is configured to detachably attach to the surface of the body covering.

In some embodiments, the device has a shape defining one of: a circle, an oval, a rectangle, a triangle, and a polygon.

In some embodiments, a circle inscribing the device has a diameter of between 5-100 mm.

In some embodiments, the device has a thickness at a thickest point thereof of between 0.25-25 mm.

In some embodiments, the resilient layer has a thickness of between 0.1-24.75 mm.

In some embodiments, the skin-proximate layer comprises an electrode comprising at least one conductive segment configured to make electrical contact with the skin of the subject.

In some embodiments, the at least one conductive segment comprises one of the following options: two or more concentric circles extending inwardly from an outer circumference of the skin-proximate layer, or at least one continuous conductive spiral extending from an outer circumference of the skin-proximate layer to a center point of the skin-proximate layer.

In some embodiments, the body covering is a garment selected from the group consisting of: shirt, pants, short pants, bodysuit, undergarment, undershirt, bra, headwear, headgear, footwear, armband, and leg band.

In some embodiments, the body covering comprises an adhesive partial body covering or dressing configured to cover an area of a body.

There is further provided, in an embodiment, a multi-segment ECG electrode, comprising: a flexible electrode pad having a forward face and a back face; one or more conductive segments defined on the forward face of the electrode pad in a specified arrangement, wherein each of the one or more conductive segments is configured to make conductive contact with a skin of a subject; and a connection point electrically connected to each of the one or more conductive segments; wherein the electrode pad comprises a through opening at a center point thereof.

In some embodiments, the electrode pad has a shape selected from the group consisting of: circular, oval, rectangular, triangular, octagonal, and polygonal.

In some embodiments, a circle inscribing the electrode pad has a diameter of between 5-100 mm.

In some embodiments, the electrode pad has a thickness at a thickest point thereof of between 0.1-1 mm.

In some embodiments, the one or more conductive segments comprise at least two conductive segments defining at least two nested shapes selected from the group consisting of: circles, ovals, rectangles, triangles, octagons, and polygons.

In some embodiments, the one or more conductive segments comprise one or more continuous spiral extending inwardly from an outer circumference of the electrode pad.

In some embodiments, the electrode pad comprises between 3-10 the conductive segments.

In some embodiments, each of the conductive segments has a width of between 0.1-5 mm.

In some embodiments, each of the conductive segments is formed by printing on the forward face using a conductive material.

In some embodiments, the electrode pad is part of a device comprising: an attachment layer configured to attach to a surface of a body covering configured to cover a body part of a subject; a skin-proximate layer configured to engage with a skin of the subject in a contact engagement at a predetermined location associated with the body part; and a resilient layer disposed between the skin-proximate layer and the attachment layer.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIGS. 1A-1E illustrate various exemplary embodiments of body coverings, e.g., shirts and pants, undershirts, underwear and bra, incorporating telemedicine devices of the present disclosure, according to various aspects of the present invention;

FIG. 2 is a block diagram illustrating an exemplary system operable to acquire and/or process health information (e.g., in conjunction with a garment system), in accordance with various aspects of the present invention;

FIGS. 3A-3B, 4A-4B, and 5A-5B show various exemplary embodiments of a telemedicine device of the present disclosure, each in side and perspective views, in accordance with various aspects of the present invention;

FIG. 6 shows a telemedicine device the present disclosure attached to a section of garment worn by a subject, in accordance with various aspects of the present invention; and

FIGS. 7A-7C shows various exemplary embodiments of a skin proximate layer or portion comprising an electrode, e.g., an ECG electrode,, in accordance with various aspects of the present invention.

### DETAILED DESCRIPTION

In accordance with some aspects of the present invention, a device, a system, and an associated method are provided incorporating bio-medical and electronic elements for extended, continuous, and/or long term monitoring, telecare, and/or telemedicine. In some embodiments, the present disclosure provides for a wearable ambulatory system incorporating bio-medical and electronic elements for continuous and/or long term physiological monitoring, telecare, and/or telemedicine, wherein the wearable platform is configured to ensure (i) user comfort during continuous and/or long-term wearing, as well as (ii) stable positioning relative to the wearer's body of monitoring, telecare and/or telemedicine elements during usage, to ensure consistent and durable monitoring and care for the duration of the usage.

Aspects and embodiments of the present disclosure are directed to a wearable system that is configured to be worn by a subject. The wearable system may comprise sensors and/or electrodes which sense biomedical and physiological information about the subject; therapy delivery elements and actuators which can deliver or administer therapy or treatment to the subject; and/or positioning and movement elements enabling monitoring of the subject position and movement and behavior analysis. At least one aspect is directed to a wearable system which includes a garment comprising one or more sensors and/or actuators and/or electrodes which sense biomedical and physiological information about the subject, analyses and diagnostics elements, and/or therapy elements which can administer therapy or treatment to the subject.

At least one aspect of the present disclosure is directed to a method of facilitating care of a subject, the method providing a wearable system configured to include a garment comprising one or more sensors and/or actuators and/or electrodes which sense biomedical and physiological information about the subject, analyses and diagnostics elements, and/or therapy elements which can administer therapy or treatment to the subject.

At least one other aspect is directed to a non-transitory computer readable medium having stored thereon program instructions. The instructions include instructions that will cause a processor to receive and process, from sensors and/or electrodes, biomedical and physiological information about a subject. The instructions may cause the processor to display information and/or issue notifications regarding at least one of a diagnosis and information about the wearable system. In various embodiments, the instructions may cause the processor to activate at least one actuator and/or operate at least one therapy element to administer therapy or treatment to the subject, based on the information received from the sensors and/or electrodes.

Aspects of the present disclosure may provide for a telemedicine device comprising, e.g., a sensor, an electrode, an actuator, a therapy element, and/or any other suitable telemedicine and therapeutic element. The telemedicine device may be configured to be attached to an article of clothing for wearing by a subject. The telemedicine device establishes physical and/or conductive contact at a specified position about the subject's skin (body) over long-term or continuous use, wherein the device is configured to maintain (i) consistent physical and/or conductive contact with the subject's skin, and (ii) consistent positioning with respect to a predetermined location on the subject's body.

In some embodiments, the present device may be used in conjunction with an article of apparel, e.g., a garment or clothing member in the form of a shirt, a pair of pants, a bodysuit, an undergarment, or any other article of apparel, such as gloves, footwear (including socks, shoes, etc.), other types of shirts (including undershirt, short sleeve or sleeveless shirts), other types of pants (including shorts or underwear), bra, hats or other headgear, arm or leg bands, belts, or any other type of apparel that is configured to cover and/or be worn on any part of a subject's body. In some embodiments, the present device may be suited for use in conjunction with undergarments, such as base layers, underwear, underclothing, brassieres, shapewear, hosiery, athletic support wear, and/or any other garment intended for wearing against the skin or as a layer that is directly exposed to a user's skin. In some embodiments, the present device suited for use in conjunction with such garments that are designed to be held to the body by means of a stretch or elastic feature or property of the garment. In some embodiments, the present device may be used in conjunction with some forms of a bodily covering or dressing, for example, an adhesive covering or dressing configured to cover an area or a portion of a body, such as the chest, abdomen, shoulder, back, any limb, or any other bodily area.

In some embodiments, the present disclosure provides for a system comprising a garment having one or more telemedicine devices of the present disclosure connected thereto and/or disposed thereon. In some embodiments, the present system further includes a port configured as a data transmission interface (e.g., wired or wireless data transmission interface), wherein the port is connected to the one or more elements using, e.g., one or more element leads and/or using a wireless connection. In some embodiments, data from the devices of the present disclosure may be transmitted, e.g., over a wired connection or wirelessly, to a remote processing module or system configured to receive and analyze the data.

By way of background, garments with integrated sensors and/or actuators and/or therapy devices configured to perform physiological monitoring and/or therapy suffer from a number of disadvantages when worn continuously or for extended periods of time. For example, physiological monitoring sensors and therapy devices depend on consistent and firm physical and/or conductive contact with a subject's skin in order to provide accurate readings. In addition, optimal and accurate monitoring may require stable positioning of certain types of sensors/devices at exact locations on a subject's body. However, reliable skin contact and positioning are difficult to ensure during long-term or continuous wearing, because of subject movement. This usually results in a negative effect on measurement/therapy results.

One possible solution is adhering the sensors/devices themselves to the subject's body, e.g., by means of adhesive backing or using another adhesive or connecting component. However, the use of adhesive-backed sensors/devices, and/or the use of other adhesive components for long term wearing may cause skin irritation and discomfort to the subject, especially in the case of young children or subjects with sensitive skin. Another possible solution is making the garment be tight-fitting, and/or providing garment regions of extra elasticity and tightness, to enhance the stability and contact of sensors with the subject's skin. However, tight-fitting garments, including those with sections having enhanced elasticity and compression, may cause discomfort and soreness to the wearer over long-term or continuous use, particularly in a garment that would ideally be worn for many hours or even whole days. In addition, even tightly worn garments often move relative to the wearer (e.g., slip or ride up), and thus cannot positively ensure a consistent positioning of the sensors.

In addition, some known garments with integrated sensors/devices rely on communications through wires, e.g., through leads or cables. This solution is typically inconvenient to the subject, because a set of long cables/leads linked between elements of the garment and a bed-side monitor may limit patient movement, and may potentially harbor infectious agents. Such solutions are also unreliable, because, with body movement of the subject, cables may disconnect or cause an associated sensor or electrode to disconnect, thereby causing interruptions, discontinuity, and data loss in the monitoring. Furthermore, when a sensor/device is disconnected or dislocated, replacement sensors/devices are not always positioned at the exact same location, thereby further compounding potential monitoring inaccuracies.

Accordingly, in some embodiments, the present disclosure provides for a device comprising one or more sensors and/or a therapy elements configured to be attached to and/or integrated into a garment. The device may be structured and shaped to cooperate with the garment, to maintain (i) consistent physical and/or conductive contact with the subject's skin, and (ii) consistent positioning with respect to a desired location on the subject's body. In some embodiments, the present disclosure provides for an article of clothing having one or more telemedicine devices attached thereto and/or integrated thereinto, that may be comfortably worn, yet provide relatively accurate and movement-insensitive measurements over a sustained period of time.

In some embodiments, the present disclosure provides for a telemedicine device and system which enable stable, continuous monitoring and/or therapy, e.g., during a hospitalization period, wherein the positioning of the sensors/therapy devices is maintained to ensure that the data is consistent and is acquired from and/or therapy delivered to exact locations on the body of the subject. The present disclosure is configured for long-term or continuous use without causing discomfort and without limiting the free movement and mobility of the subject.

In some aspects, a wearable health monitoring and therapy system of the present disclosure comprises a garment having telemedicine devices integrated thereinto, e.g., into a wicking fabric. When the wearable system is worn by a user in accordance with some aspects of the present invention, the integrated telemedicine devices from an array of telemedicine devices in a predetermined arrangement over one or more body parts. In some aspects, the one or more telemedicine devices of the wearable system are arranged in an array of telemedicine devices which is disposed with respect to a specified location in a body region of the user when the system is worn by the user. In some embodiments, each of the telemedicine devices is in communication with a local and/or remote processing module of the wearable system. The processing module typically comprises processor(s) comprising processing and control circuitry, e.g. microprocessors and or microcontrollers, which are configured to receive, process, control, monitor and transmit data and/or signals to and from each one of the telemedicine devices in the wearable system.

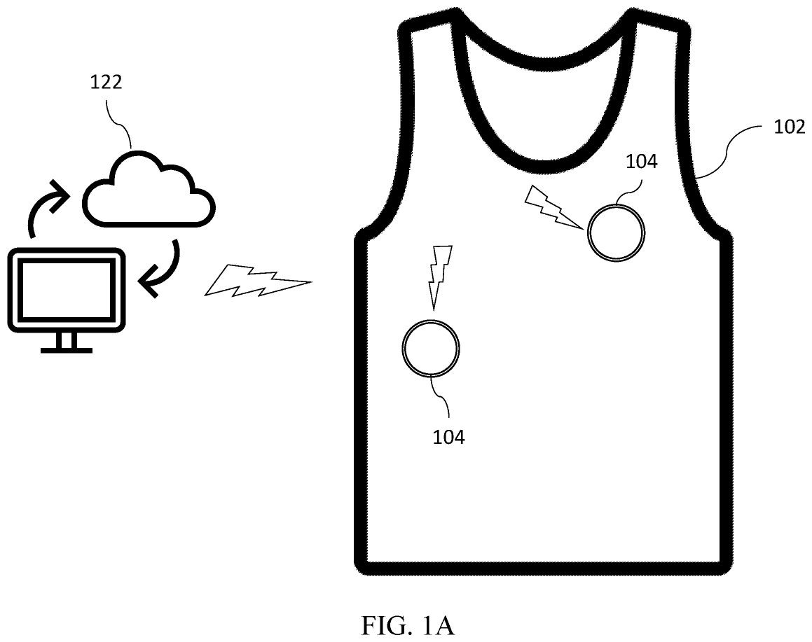

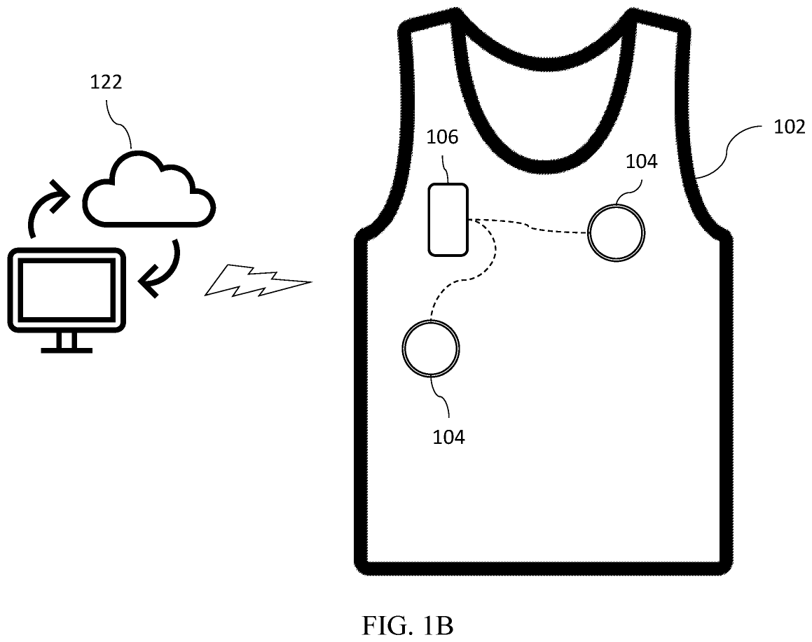

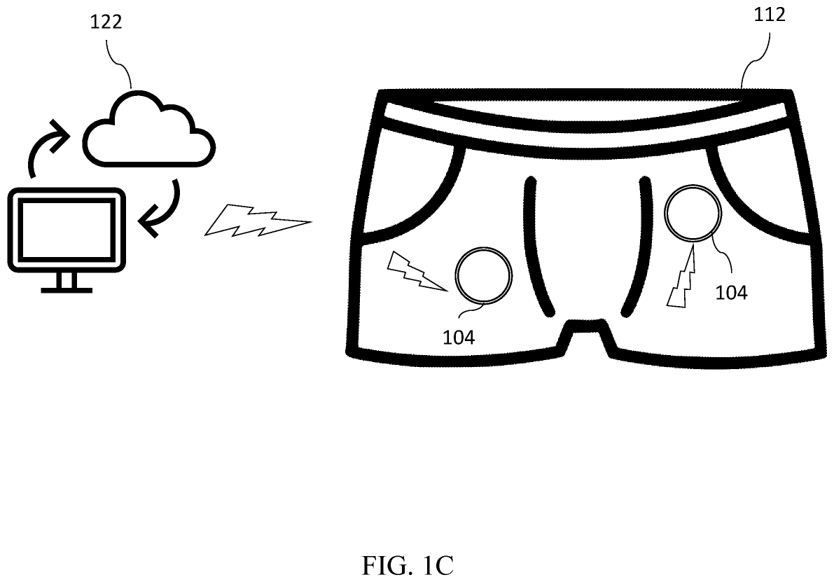

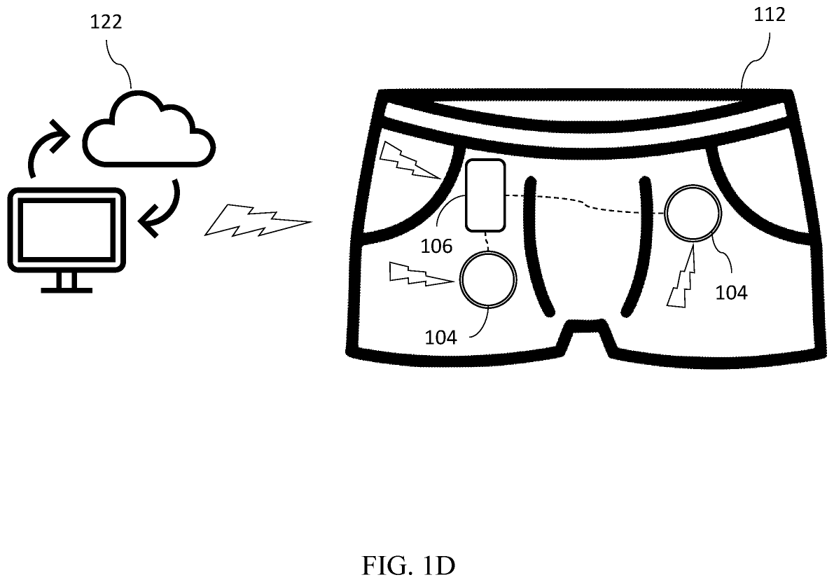

FIGS. 1A-1E illustrate various exemplary embodiments of garments, e.g., an undershirt 102 and underpants 112, according to various aspects of the present invention. The exemplary garments 102, 112 comprise one or more telemedicine devices 104 of the present disclosure, each comprising one or more of a variety of sensors/therapy elements, and are attached and/or integrated into a garment. Such garment may, for example, generally correspond to articles of clothing that a person may wear throughout the course of typical life activities (e.g., sleeping, eating, walking, working, exercising, watching television, doing household chores, traveling, socializing, etc.). In some embodiments, such garments may be, but are not necessarily, health-care specific garments (e.g., a hospital gown, a specific garment worn in a hospital in a control environment for a specific test, etc.). For example and without limitation, such garments may comprise: a full or partial body suit, a long-sleeve shirt, short-sleeve shirt, sleeveless shirt, undershirt, pants, shorts, underwear, socks, swimwear, a sports bra, undergarments, a wet suit, thermal wear, a glove, and so on. Various aspects of the present invention may also apply to motorists to monitor their health while driving and/or to protective gear, for example, as may be worn during performing hazardous jobs (e.g., a hard hat, body armor, etc.), participating in risky recreational, sporting, law enforcement, or military activities, etc.

In some embodiments, garments 102, 112 of the present disclosure may be formed in whole or part with the appropriate form-fitting elasticity, e.g., a moderate amount of elasticity, so as to ensure the application of sufficient force to maintain telemedicine devices 104 in constant contact with the skin of the subject wearing the garment. However, garments 102, 112 of the present disclosure are not formed with excessive elasticity and/or compression features, so as to render garments 102, 112 uncomfortable for wearing during long-term or continuous use.

FIGS. 1A and 1C shows exemplary garments 102, 112 (e.g., a shirt and pants or an undershirt and underwear) as part of a wearable system of the present disclosure. Garments 102, 112 comprise a plurality of telemedicine devices 104 disposed at locations which may be specified for the measuring of physiological parameters, e.g., ECG, and/or delivery of specified therapy, e.g., insulin injection or defibrillation shock.

As shown in FIGS. 1B and 1D, in some embodiments, each of the telemedicine devices 104 may be conductively coupled (e.g., utilizing conductive fiber and/or wire) to a port 106. In an exemplary scenario, port 106 may comprise a communication hub by which measurement circuitry may electrically access the sensors/therapy elements in telemedicine devices 104, for analysis and/or communication to, e.g., a remote system, such as remote system 122. In some embodiments, port 106 may also comprise a power supply (e.g., a battery) or be conductively coupled to a power supply (e.g., a power supply integrated into the garments 102 and/or off-garment.

FIG. 1E shows an exemplary partial body covering or dressing 114, which may be an adhesive covering or dressing. Partial body covering 114 comprises at least one telemedicine devices 104 disposed at a location which may be specified for the measuring of physiological parameters, e.g., ECG, and/or delivery of specified therapy, e.g., insulin injection or defibrillation shock.

In some embodiments, telemedicine device 104 of the present disclosure may comprise one or more integrated sensors which may comprise characteristics of any of a variety of different types of sensors, signals from which may be analyzed to ascertain health. Such sensors may, for example, comprise physiological sensors that operate to monitor physiological characteristics of a or subject, e.g.: heart monitoring sensors, heart-rate monitoring sensors, electrocardiogram (ECG) sensors, body temperature sensors, breath-rate/respiration sensors (e.g., pressure-based and/or acoustic-based), skin conductivity sensors, oxygen saturation sensors, blood perfusion sensors, skin humidity sensors, electroencephalograph (EEG) sensors, and/or muscle activity sensors.

In some embodiments, such sensors may also comprise non-physiological sensors that operate to monitor characteristics other than physiological characteristics of the subject. Such non-physiological sensors may, for example, comprise environmental sensors that operate to monitor aspects of the environment in which a subject is performing a task. For example and without limitation, such environmental sensors may comprise: gas concentration sensors (e.g. carbon monoxide), radiation sensors, electromagnetic field sensors, air temperature sensors, air speed sensors, humidity sensors, air oxygen level sensors, barometric pressure sensors, altitude/elevation sensors, precipitation sensors, light sensors, movement sensors, e.g. multi-axes accelerometers, location sensors/systems (e.g., global positioning system (GPS) sensors, ranging and direction sensors, terrestrial triangulation sensors/systems (e.g., cellular communication system based, premises based, campus based, etc.), time sensors (e.g., time change and/or absolute time), orientation sensors, etc. Such non-physiological sensors may also, for example, comprise situational sensors that operate to monitor characteristics of a physical situation (e.g., a task or activity) in which the subject is engaged. Such situation (or activity) sensors may, for example and without limitation, comprise: weight sensors, impact sensors, force sensors, pressure sensors, accelerometers, inclinometers, motion sensors, speed and/or velocity sensors, etc.

In some embodiments, telemedicine device 104 of the present disclosure may comprise one or more integrated therapy elements, e.g., elements configured to administer to deliver a specific therapy or treatment to the subject at a predetermined location. For example, such therapy element may be a defibrillator configured to deliver defibrillating energy to the subject, e.g., to convert heart arrhythmia to a normal rhythm. In other examples such therapy element may be configured to deliver a dose of medication or a specified substance, such as insulin, to the subject. In some embodiments, telemedicine device 104 may comprise invasive elements, such as sensors and therapy elements which include probes or wires that are insertable into a subject's skin.

In some embodiments, a telemedicine device may be attached to and/or integrated or incorporated into an article apparel 102, 112 or the present disclosure in a variety of manners. For example, a telemedicine device of the present disclosure may be snapped, adhered and/or sewn during manufacturing and/or to a completed garment. For example, a telemedicine device 104 of the present disclosure may be secured to the inside of the garment using, e.g., any suitable manner of attachment, e.g., by the use of adhesives, hook and loop attachment, and the like.

In some embodiments, a garment 102, 112 of the present disclosure may comprise one or more telemedicine devices 104, e.g., positioned in one or more desired locations, which may be customized to a particular subject. Such a scenario provides flexibility for positioning particular placement-sensitive telemedicine devices 104 (e.g., comprising ECG sensors) at locations suited for a particular subject. In other embodiments, a garment 102, 112 of the present disclosure may comprise one or more telemedicine devices 104 which may be formed into the garment while the garment is being formed. In such a scenario, additional, e.g., location-critical telemedicine devices 104 may be added later for a particular subject. In another exemplary scenario, the specifications for a garment and telemedicine devices placement may be customized for a particular subject, and such specifications may then be provided to a manufacturer of the garment for customized production of the garment.

In some embodiments, telemedicine devices 104 may comprise various sensors/therapy elements which may be shaped, positioned or formed in any of a variety of manners, depending on the nature of the particular sensor/therapy element. For example, some sensor surfaces (e.g., electrodes) may generally comprise conductive material. For example, an electrode may comprise a metallic surface exposed for subject contact. In another example, an electrode may be formed from conductive plastic (or another material) that may be integrated into various molded components of the mobile communication device. For example, various conductive plastics (e.g., graphite-impregnated plastic or the like) may provide sufficient conductivity for an electrode to perform adequately. It should be recognized that the scope of various aspects of the present invention should not be limited by characteristics of particular electrodes or electrode placements unless explicitly claimed. Electrodes (or other sensors) may be shaped, positioned or formed with various physical features to enhance collection of cardiovascular information from a subject. For example and without limitation, an electrode may comprise one or more projections to enhance conductive contact with a subject. Also, an electrode may comprise one or more depressions or indentations to enhance conductive contact with a subject.

In some embodiments, telemedicine devices 104 may be self-contained devices comprising, e.g., their own respective power supply and their own respective communication circuitry. For example, one or more of telemedicine devices 104 may comprises a transceiver configured to transmit and receive signals and/or data to and from a remote computing system, e.g., computing system 122, which may be cloud-based. In some embodiments, telemedicine devices 104 may operate to wirelessly communicate information over a wireless connection.

In other cases, telemedicine devices 104 may require connecting to a local port 106 (shown in FIGS. 1B and 1D) operationally coupled to telemedicine devices 104 through leads (e.g., wire leads, conductive fiber leads, etc.) which may be woven into the garment, run via garment seams, etc. In some embodiments, port 106 may be configured to send and receive signals and/or data to and from the various in-garment sensors/therapy elements. In some embodiments, port 106 may comprise processing capabilities configured to process signals and/or data and prepare information describing such signals for transmission (e.g., characterizing such signals in terms of a set of voltage potential differences). In some embodiments, port 106 comprises a transceiver configured to transmit and receive raw and/or processed signals and/or data a remote computing system, e.g., computing system 122, which may be cloud-based.

In some embodiments, local port 106 may comprise, for example, a portable computing device worn on the body, a tablet computer, a smartphone, etc. A portable computing device or an accessory thereof may be configured to continuously measure one or more physiological signals of a user, based on continuous communication telemedicine devices 104. The portable computing device may have loaded onto (e.g. onto a non-transitory computer readable medium of the computing device) and executing thereon (e.g. by a processor of the computing device) an application for one or more of receiving the continuously measured physiological signal(s), analyzing the physiological signal(s), sending the physiological signal(s) to a remote computer for further analysis and storage, and displaying to the user analysis of the physiological signal(s). for example, heart rate may be measured by one or more electrodes provided by telemedicine devices 104. In response to the continuous measurement and recordation of the heart rate of the user, parameters such as heart rate (HR), heart rate variability (R-R variability or HRV), and heart rate turbulence (HRT) may be determined. These parameters and further parameters may be analyzed to detect and/or predict one or more of atrial fibrillation, tachycardia, bradycardia, bigeminy, trigeminy, heart attack, stroke, or other similar conditions. A quantitative heart health score may also be generated from the determined parameters. The biometric data may in turn be uploaded onto a remote server, e.g., remote system 122, where one or more cardiac technicians or cardiac specialists may analyze the biometric data and provide ECG interpretations, diagnoses, recommendations such as lifestyle recommendations, and/or goals such as lifestyle goals for subject.

In some embodiments, a wearable system of the present disclosure may incorporate a plurality of ECG sensors, electrodes, multi-axes motion sensors, and/or positioning and location sensors. In some embodiments, the system measures continuously the signals received from these sensors generated by the subject's body. These signals and data are continuously transmitted to a processing unit for processing and are recorded on a memory unit, e.g., a built in memory or a removable memory such as SD card. The measured signals are continuously processed and analyzed by a machine-learning engine, a signal data processing algorithm which continuously generates a reference signal for filtering noise and distinguishing signals that do not conform to, or deviate from, a reference signal. In addition, the algorithm analyses these deviated signals in comparison with data received from the movement sensors enabling correlation of the received signals with the movement data, and thus creating a behavioral model of the subject's movement. This, in turn, provides for the ability to isolate the effects of the subject's movements from the cardiac signals, to indicate the subject's movements behavior, and to achieve an improved diagnostics of the subject's cardiac state. In addition, this enables the present system to establish a cardiac signature specific to the subject. With time, as more data is captured and accumulated from the wearable unit sensors, the quality of the reference signal and the behavioral model determined by the machine learning algorithm is improved.

Accordingly, in some embodiments, a wearable system of the present disclosure may provide for ambulatory measurement of cardiac activity in a subject, to facilitate extended and/or continuous health monitoring, e.g., of subjects with a history of cardiovascular conditions. However, a major challenge of ambulatory monitoring is the effect on cardiac activity signals (e.g., ECG signals) by artifacts generated through body motion of the subject being monitored. Accordingly, in some embodiments, the present disclosure provides for isolating the effects of noise (e.g., motion artifact) mixed with a cardiac activity signal.

In some embodiments, a wearable system of the present disclosure may comprise one or motion sensors to determine at least some of body motion, movement, position, and/or orientation. For example, motion sensors may be used to determine whether a subject's body is stationary or in motion, and whether the subject is supine, seated, standing, walking, running, bending over, etc. In some embodiments, the motion sensors may comprise, e.g., one or more multi-axis accelerometers, pedometers, electromechanical motion detectors, etc.

In some embodiments, the ECG signals may be correlated with changes in the subject's movement and/or position. In some embodiments, the present disclosure provides for predictive models which can estimate a risk of a cardiac event in a subject based on measured ECG signals and a motion or activity status of a subject.

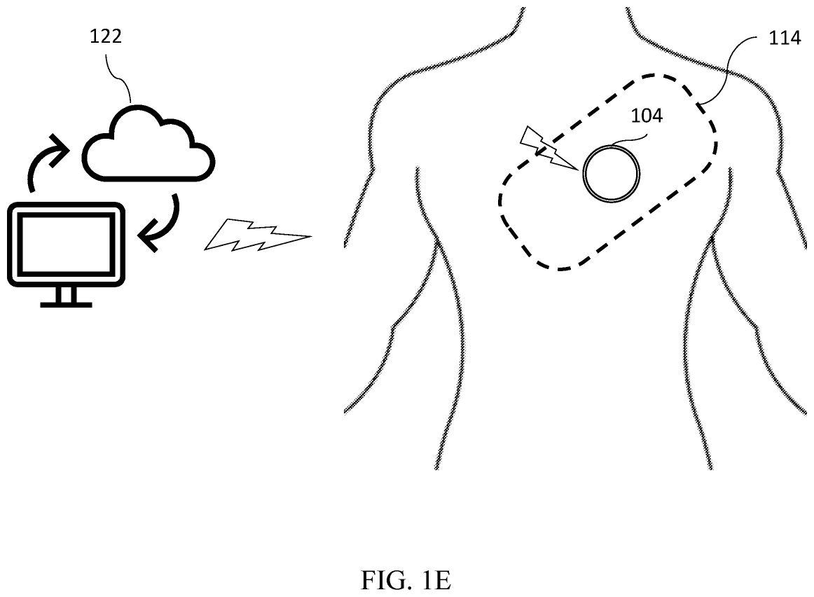

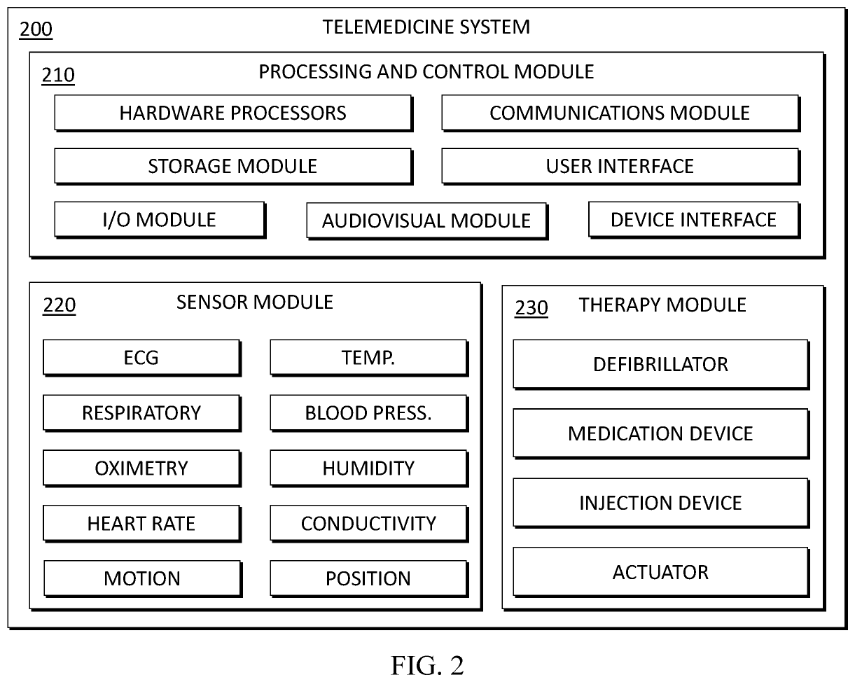

FIG. 2 is a block diagram illustrating an exemplary telemedicine system 200 of the present disclosure, operable to acquire and/or process health information (e.g., in conjunction with a garment system), in accordance with various aspects of the present invention.

In some embodiments, system 200 may be implemented in a single device. Such a single device may, for example, be integrated into a garment (e.g., the same one or more garments in which the sensors of interest are integrated). In some embodiments, such a device may be a dedicated health-monitoring device, e.g., a mobile or desktop computing device (e.g., a smartphone, a desktop or laptop computer, a tablet) operatively coupled to telemedicine devices integrated into a garment of the present disclosure.

In other examples, system 200 may comprise a centralized computing device, e.g., located at a health-care facility, emergency vehicle, or physician office), operatively coupled to telemedicine devices integrated into a garment of the present disclosure. The various modules of the system 200 may be implemented in hardware, software, or any combination of hardware and software. In some embodiments, system 200 may be implemented in a distributed system (e.g., with dispersed components), some of which may be implemented using cloud computing platforms and/or edge computing network and/or a hybrid platform. For example, various modules of the system 200 may be integrated in a plurality of different devices and computers. For example, a first set of modules of the system 200 may be integrated in a garment such as telemedicine devices 104 in garments 102, 112 in FIGS. 1A-1E, and a second set of modules of the system 200 may be implemented in one or more remote computing devices.

In some embodiments, system 200 may comprise a processing and control module 210 implemented as a remote computing system, e.g., system 122 in FIGS. 1A-1E, that operates to acquire sensor signals and/or information from a sensor module 220, which may comprise, e.g., one or more telemedicine devices 104 of the present disclosure, each comprising one or more sensors and/or electrodes. In some embodiments, processing and control module 210 may operate one or more therapy elements in therapy module 230, e.g., a defibrillator, injection device, and/or medication device, to administer therapy to the subject.

In some embodiments, processing and control module 210 comprises one or more hardware processor(s), one or more controller(s) one or more memory storage device, comprising a random-access memory (RAM) and one or more non-transitory computer-readable storage device(s), a communications module, input output, digital and analogue interfaces, display and a user interface. The storage device(s) may have stored thereon program instructions and/or components configured to operate the hardware processor(s) of processing and control module 210. The program instructions may include one or more software modules and/or an operating system having various software components and/or drivers for controlling and managing general system tasks (e.g., memory management, storage device control, power management, etc.), and facilitating communication between various hardware and software components. In some embodiments, processing and control module 210 may be configured to receive updates to the program instructions wirelessly.

Processing and control module 210 may operate to perform any or all functionality with regard to the acquisition, analysis and/or processing of data and/or signals and/or information from various telemedicine devices in sensor module 220, and/or with regard to operating the devices in therapy module 230. In some embodiments, processing and control module 210 may operate to receive a raw unprocessed analog signal from a telemedicine device (e.g., an analog voltage signal from an electrode). In other exemplary scenarios the processing and control module 210 may operate to receive one or more signals from a sensor communicating data from such sensor. In some embodiments, System 200 may comprise one or more communications modules via which the system 200 may communicate with a plurality of other systems utilizing wired and/or wireless communication, e.g., any short-or long-range wireless communications protocol, such as wi-fi, Bluetooth, ZigBee, cellular communications, any radiofrequency protocol, etc.

The manner in which the health information acquisition module acquires such information depends on the nature of the source from which such information is obtained and/or the nature of the communication network(s) communicatively coupling system 200 to such source. For example, in various scenarios, processing and control module 210 may operate to receive such information from a wired and/or wireless data communication network, a wired and/or wireless telecommunication network, etc.

System 200 additionally comprises one or more user interface modules. Such user interface module(s) may operate to communicate information with a user (e.g., receive input information from such user and/or output information to the user, including audio visual interface).

In some embodiments, system 200 may receive electrical power from any of a variety of sources. For example, in a scenario where at least one module of system 200 is integrated into a garment (e.g., one or more of garments in which sensors are integrated), a power supply for such at least one module may be integrated into the garment and/or a power supply connection may be integrated into such garment via which power may be supplied to such at least one module by a source external to such garment. In some embodiments, where at least one module of system 200 is integrated into a personal computing device (e.g., a smartphone, tablet, etc.), a power supply for such at least one module may be located in such device. In some embodiments, where at least one module of system 200 is integrated into a garment (e.g., one or more of garments in which sensors are integrated) and at least one module of system 200 is integrated into a separate computing device, system 200 may comprise independent power supplies for each part thereof, or may comprise a single power supply for all part thereof.

In some embodiments, sensor module 220 may comprise one or more telemedicine devices 104, each comprising one or more sensors and/or electrodes and/or actuators configured to measure one or more physiological parameters and/or activate therapy devices, such as, but not limited to:
heart monitoring sensors,
heart-rate monitoring sensors,
electrocardiogram (ECG) sensors,
body temperature sensors,
breath-rate/respiration sensors (e.g., pressure-based and/or acoustic-based),
skin conductivity sensors,
oxygen saturation sensors,
blood perfusion sensors,
blood pressure sensors,
skin humidity sensors,
electroencephalograph (EEG) sensors, and/or
muscle activity sensors.

In some embodiments, sensor module 220 may comprise one or more telemedicine devices 104, each comprising one or more sensors and/or electrodes and/or actuators configured to measure non-physiological parameters, e.g., configured to operate to monitor characteristics other than physiological characteristics of the subject and/or activate therapy devices, such as, but not limited to:
Air temperature sensors,
air speed sensors,
humidity sensors,
air oxygen level sensors,
barometric pressure sensors,
altitude/elevation sensors,
precipitation sensors,
light sensors,
location sensors/systems (e.g., global positioning system (GPS) sensors,
time sensors,
weight sensors,
impact sensors,
force sensors,
pressure sensors,
magnetometer,
gas concentration sensors, e.g. carbon monoxide
radiation sensors,
electromagnetic field sensors,
accelerometers,
inclinometers,
movements and motion sensors,
range and direction sensors.
gyroscope and dead reckoning
speed and/or velocity sensors.
control elements.
Actuators.
converters, analog to digital and digital to analog.
audio visual means.

In some embodiments, therapy module 230 may comprise one or more therapy elements configured to administer therapy to a subject, e.g., insulin injection, defibrillation shock, and/or a dose of medicine.

In some embodiments, system 200 may comprise a port, e.g., port 106 (shown in FIGS. 1B and 1D) operationally coupled to telemedicine devices 104 within sensor module 220. In some embodiments, port 106 may be configured to receive signals and/or data from the various in-garment sensors and electrodes. In some embodiments, port 106 may comprise processing capabilities configured to process such signals and/or data and prepare information describing such signals for transmission (e.g., characterizing such signals in terms of a set of voltage potential differences). In some embodiments, port 106 may comprise controlling capabilities configured to control such sensors and therapy elements. In some embodiments, port 106 comprises a transceiver configured to transmit and receive raw and/or processed signals and/or data to and from a remote computing system, e.g., processing and control module 210 of system 200.

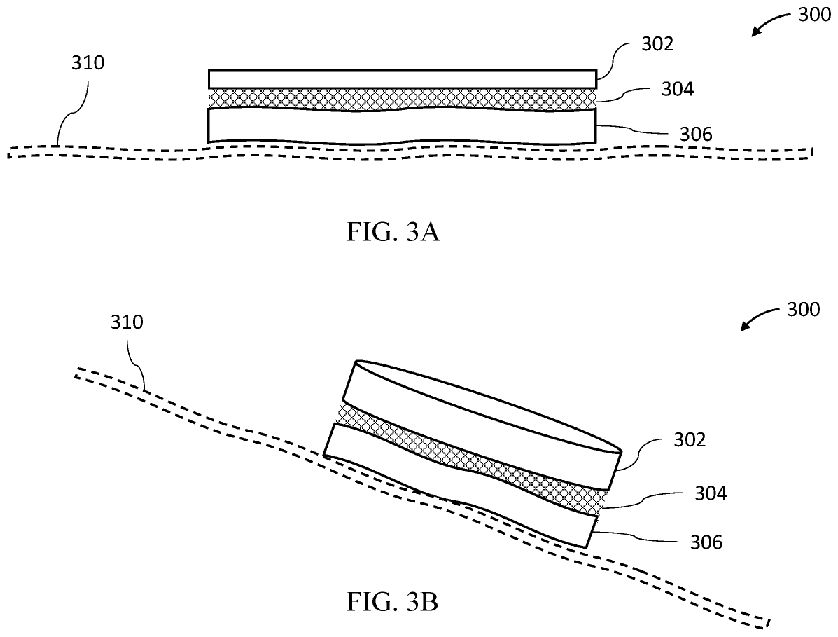

FIGS. 3A-3B, 4A-4B, and 5A-5B show various exemplary embodiments of a telemedicine device 300, 400, 500, 600, each in side and perspective views, in accordance with some embodiments of the present disclosure.

In some embodiments, telemedicine device 300, 400, 500, 600 comprise a device comprising a sensor or electrode configured to be attached to and/or integrated into a garment, such as garments 102, 112 in FIGS. 1A-1E. device 300, 400, 500, 600 may be structured and shaped to work in cooperation with the garment, to maintain (i) consistent physical and/or conductive contact with the subject's skin 310, 410, 510, 610 and (ii) consistent positioning with respect to a desired location on the subject's body.

In some embodiments, telemedicine device 300, 400, 500, 600 may be attached to and/or integrated or incorporated into a garment of the present disclosure in a variety of manners. For example, a telemedicine device 300, 400, 500, 600 of the present disclosure may be snapped, adhered and/or sewn during manufacturing and/or to a completed garment. For example, a telemedicine device 300, 400, 500, 600 of the present disclosure may be secured to an inside surface of a garment using, e.g., any suitable manner of attachment, e.g., by the use of adhesives.

In some embodiments, telemedicine device 300, 400, 500, 600 may be shaped and dimensioned as per their usage and operation requirements, to be disposed between a garment of the present disclosure and a skin 310, 410, 510, 610 of a subject in an unobtrusive manner. In some embodiments, device 300, 400, 500, 600 may have a circular, oval, rectangular, triangular, octagonal, and/or any other polygonal shape. In some embodiments, a circle inscribing device 300, 400, 500, 600 may have a small diameter of between 1-5 mm, a mid-size diameter of between 5-10 mm, or a larger diameter of between 10-100 mm. In some embodiments, device 300, 400, 500, 600 has a thickness at a thickest point thereof of between fractions of a millimeter (for example, between 0.25-1 mm), and up to 25 mm. Though other sizes, diameters and or thickness, may prevail, depending on requirements.

In some embodiments, telemedicine device 300, 400, 500, 600 may comprise a skin proximate layer or portion 306, 406, 506, 606 comprising one or more sensors and/or therapy elements, wherein a forward surface of skin proximate layer 306, 406, 506, 606 is configured to engage a subject's skin in proximate, contact, and/or electrically-conductive engagement. In some embodiments, proximate layer 306, 406, 506 may comprise a flexible substrate configured for external application to the skin at a body site, there to establish and maintain contact with a subject's skin 310, 410, 510, 610 over the entirety or at least a portion of the surface area of proximate layer 306, 406, 506. In some embodiments, the contact may comprise electrically-conductive engagement with the skin, which enables measurements of a plurality of physiological parameters by one or more sensors, and/or to enable the administering of a specified therapy to the subject by one or more therapy elements. In some embodiments, proximate layer 306, 406, 506, 606 may be configured to establish and maintain physical and/or conductive contact with a subject's skin 310, 410, 510, 610 over at least a portion of a surface area of proximate layer 306, 406, 506, 606. In some embodiments, proximate layer 306, 406, 506 may be shaped, positioned or formed in any of a variety of manners, depending on the nature of the particular sensor/therapy element associated with proximate layer 306, 406, 506, 606. For example, in some embodiments, proximate layer 306, 406, 506, 606 may generally comprise a conductive material, e.g., a metallic surface exposed for subject contact. In another example, proximate layer 306, 406, 506, 606 may be formed from conductive plastic (e.g., graphite-impregnated plastic or the like), which may provide sufficient conductivity for proximate layer 306, 406, 506, 606 to perform adequately. In some embodiments, proximate layer 306, 406, 506, 606 may be at least partially deformable to allow proximate layer 306, 406, 506, 606 to generally conform to the surface contours of the subject's anatomy. In some embodiments, proximate layer 306, 406, 506, 606 may be formed from one or more of silicone, polyethylene, polyvinyl chloride (“PVC”), polypropylene, nylon, polyurethane, polycarbonate, and other plastics and or steel, aluminum, copper, silver, gold, and other metals.

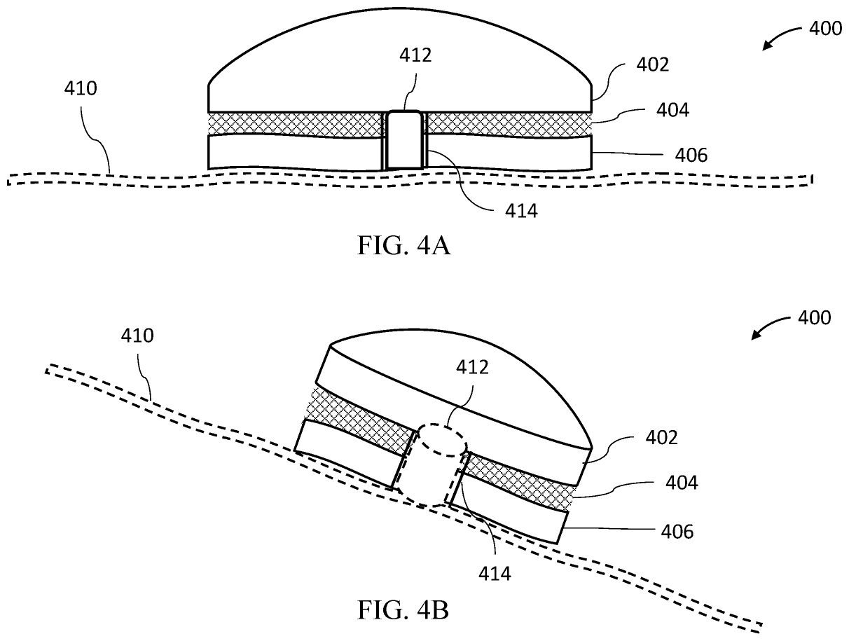

In some embodiments, with reference to FIG. 4A, exemplary telemedicine device 104 may comprise a medication and/or therapy delivery element comprising, e.g., a canister 412 for storing a specified amount of medication and/or therapy. The medication and/or therapy delivery element is shown in conjunction with the exemplary telemedicine device 400, however, it may be included in any one of exemplary telemedicine devices 300, 400, 500, 600.

In some embodiments, canister 412 may be placed, e.g., within an opening or cavity 414 within attachment layer 302, 402, 502, 602 and/or resilient layer 304, 404, 504, 604. In some embodiments, the opening 414 may be a through opening through proximate layer 306, 406, 506, 606, wherein a medication and/or therapy stored in canister 412 may be delivered to a subject through opening 414, using any suitable medication delivery method.

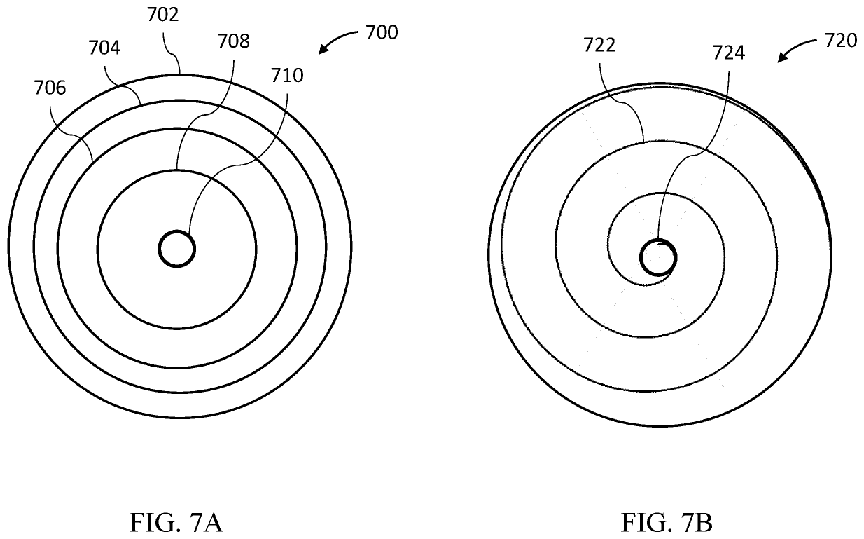

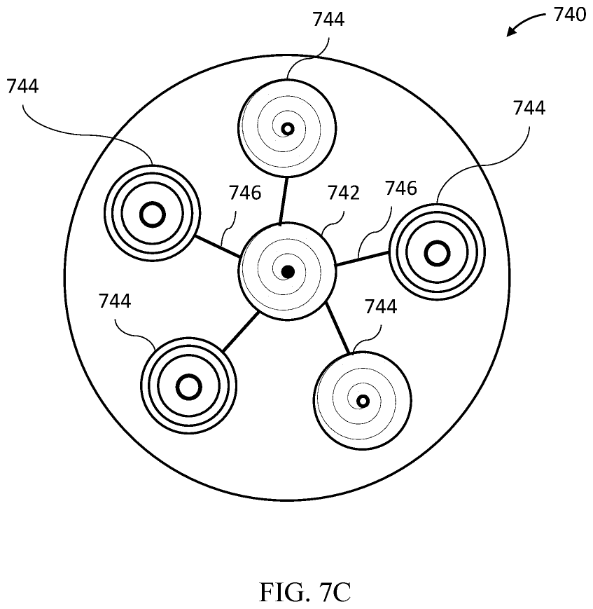

FIGS. 7A-7B illustrate exemplary embodiments of a skin proximate layer or portion 306, 406, 506, 606 comprising an electrode 700, 720, 740, e.g., an ECG electrode, according to some embodiments.

By way of background, commonly-used sticker-based electrodes are not suitable for extended and/or continuous durable cardiac activity monitoring. Over time and under body motion of the monitored subject, these electrodes may detach from the body, and may need to be replaced with new electrodes, thus causing disruption in the continuous extended monitoring. In addition, replacement electrodes may not be located in the exact location of the detached replaced electrode, resulting in inconsistency in the obtained signals.

Accordingly, in some embodiments, an electrode 700, 720, 740 of the present disclosure may comprise a thin, sub-millimeter, flexible layer of insulating material, on which may be printed segments of a conductive material. The dimensions of the electrode 700, 720, 740 and conductive segments and its pattern may be determined depending on the signal to be captured and the expected extent of body movement by the wearer.

An electrode 700, 720, 740 of the present disclosure may comprise multiple conductive segments and/or traces, wherein each may correspond to a separate terminal. For purposes of the present disclosure, a segment may be defined as a conductive area on an electrode 700, 720, 740 that is intended to make electrical and/or conductive contact with a subject's body (e.g., skin). For purposes of the present disclosure, a conductive trace may define a generally narrow conductive strip, e.g., a circle, an arc, a straight line, etc.

In some embodiments, multi-segment and/or multi-trace electrodes 700, 720, 740 may be used in producing low-noise ECG signals. A major source of noise in ECG signals is baseline noise generated in the electrode. The baseline noise is low frequency noise that appears as an undulating baseline upon which the ECG rides. Baseline noise is attributable to motion and deformation of the electrode and its associated conductive material, and results from low frequency events such as patient respiration and patient motion. As a result, the magnitude of baseline noise tends to increase with exercise. However, many important ECG measurements must be made during motion and/or physical exertion.

Accordingly, in some embodiments, the present invention features a multi-segment and/or multi-trace electrode 700, 720, 740. The electrode 700, 720, 740 includes a flexible base-pad, comprising a plurality of conductive segments or traces defined on a surface of the base-pad. The base-pad may include a region that defines a connection point, and conductive leads may extend from each of the segments to the connection point. In some embodiments, the connection point may comprise a through hole in the base-pad, through which conductive leads may pass. In some embodiments, the segments and/or traces may be formed by printing with a conductive material on the surface of the flexible base-pad. The conductive material may be, for example, silver-chloride ink, copper, etc.

For example, electrode 700 in FIG. 7A may comprise a skin-facing surface of a body-proximate layer 306, 406, 506, 606 of a telemedicine device of the present disclosure. Electrode 700 may comprise a base-pad formed of an insulating, flexible film, such as polyester film, which may have a thickness of, e.g., between 0.20-0.35 mm.

The base-pad comprises a plurality of concentric conductive circles, e.g., circles 702-708, each defining a conductive trace intended to make electrical and/or conductive contact with a subject's body (e.g., skin).

In some embodiments, the electrode may comprise other concentric and/or nested shapes, e.g., oval, ellipse, square, rectangular, or any other polygonal shape. Each conductive circle 702-708 may be connected to a connection point (not shown) of electrode 700 through, e.g., conductive leads which may extend from each of circles 702-708. In some embodiments, the connection point may be connected to the conductive circles through a hole 710, through which the conductive leads may pass. In some embodiments, conductive circles 702-708 may be formed by printing with a conductive material on the surface of the flexible base-pad. In some embodiments, hole 710 may be configured for cooperating with a medication and/or therapy delivery element, e.g., canister 412 in FIG. 4A. In some embodiments, canister 412 may work in cooperation with a through opening or hole 710, wherein a medication and/or therapy stored in canister 412 may be delivered to a subject through opening 710, using any suitable medication delivery method.

In some embodiments, electrode 700 may have a total diameter of between., e.g., 15-55 mm. Electrode 700 may comprise, e.g., between 3 and 10 concentric circles, spaced radially apart e.g., between 0.5-5 mm. In some embodiments, each conductive circle may comprise a conductive trace having a width of between, e.g., 0.5-3.5 mm.

Another example is electrode 720 in FIG. 7B, which may comprise a skin-facing surface of a body-proximate layer 306, 406, 506, 606 of a telemedicine device of the present disclosure. Electrode 720 may comprise a base-pad formed of an insulating, flexible film, such as polyester film, which may have a thickness of, e.g., between 0.20-0.35 mm.

The base-pad comprises one or more continuous spiral conductive trace 722, defining a conductive trace intended to make electrical and/or conductive contact with a subject's body (e.g., skin). Conductive trace 720 may be connected to a connection point (not shown) of electrode 720 through, e.g., conductive leads which may extend from each conductive trace 722. In some embodiments, the connection point may be connected to the conductive trace 722 through a hole 724, through which the conductive leads may pass. In some embodiments, conductive trace 722 may be formed by printing with a conductive material on the surface of the flexible base-pad.

In some embodiments, electrode 720 may have a total diameter of between., e.g., 15-55 mm. Electrode 720 may comprise, e.g., between 1 and 3 continuous spiral conductive traces, spaced radially apart e.g., between 0.5-5 mm. In some embodiments, each conductive circle may comprise a conductive trace having a width of between, e.g., 0.5-3.5 mm.

FIG. 7C illustrates another example of a cluster electrode 740, which may comprise a skin-facing surface of a body-proximate layer 306, 406, 506, 606 of a telemedicine device of the present disclosure. Cluster electrode 740 may comprise a base-pad formed of an insulating, flexible film, such as polyester film, which may have a thickness of, e.g., between 0.20-0.35 mm.

Cluster electrode 740 may be intended to provide a greater area coverage, and thus may have a total diameter of, e.g., between 50 and 200 mm, or larger. Cluster electrode 740 may comprise a plurality of sub-electrodes, e.g., a central sub-electrode 742 surrounded by multiple external sub-electrodes 742. Each of the central and external sub-electrodes may be configured as a multi-segment and/or multi-trace electrode, e.g., electrode 700 in FIG. 7A and/or electrode 720 in FIG. 7B. Thus, each sub-electrode may comprise, e.g., a plurality of concentric conductive circles and/or one or more continuous spiral conductive traces. Each sub-electrode may be connected to the central via a lead 746.

With reference back to FIGS. 3A-3B, 4A-4B, and 5A-5B, in some embodiments, telemedicine device 300, 400, 500, 600 may comprise a soft and flexible resilient layer or portion 304, 404, 504, 604 which may be an intermediate layer sandwiched between attachment layer 302, 402, 502, 602 and proximate layer 306, 406, 506. In some embodiments, resilient layer 304, 404, 504, 604 may comprise a layer comprising one or more resilient materials of different thicknesses and characteristics, configured to cooperate with the garment to exert a force which tends to secure a positioning of telemedicine device 300, 400, 500, 600 at a predetermined location on the subject's body. In some embodiments, resilient layer 304, 404, 504 has a thickness of between 1.00-24.5 mm.

Accordingly, in some embodiments, a garment of the present disclosure may be formed with a predetermined form-fitting elasticity, suited to ensure the application of a predetermined amount of force to one or more attached telemedicine device 300, 400, 500, 600, so as to constant contact with the skin 310, 410, 510, 610 of the subject wearing the garment. However, a garment of the present disclosure may not be formed with excessive elasticity and/or compression features, so as to allow the garment to be worn by a subject comfortably over extended periods of time. Accordingly, in some embodiments, resilient layer 304, 404, 504 cooperates with the garment to exert additional force locally with respect to each telemedicine device 300, 400, 500, 600 so as to enhance locally the amount of resilient force applied specifically to each telemedicine device 300, 400, 500, 600.

In some embodiments, resilient layer 304, 404, 504, 604 may comprise one or more resilient materials, arranged in any suitable configuration, e.g., one or more layers and the like. In some embodiments, resilient layer 304, 404, 504, 604 may be fashioned of any suitable resilient and/or elastomeric material, e.g., silicone rubber, rubber, polyurethane, a variety of foam materials, and the like. In some embodiments, resilient layer 304, 404, 504, 604 may combine two or more resilient materials having each varying properties, e.g., elastic modulus.

In some embodiments, resilient layer 304, 404, 504, 604 may comprise one or more resilient materials, arranged in any suitable configuration, e.g., one or more layers and the like. In some embodiments, resilient layer 304, 404, 504, 604 may comprise a canister 305, 405, 505, 605 of appropriate size for e.g., medicine, medical gel or other materials as per application.

In some embodiments, telemedicine device 300, 400, 500, 600 may comprise an attachment layer or portions, e.g., layer 302, 402, 502, 602, configured to attach telemedicine device 300, 400, 500, 600 to a garment. In some embodiments, attachment layer may comprise adhesive backing layer. In some embodiments, the adhesive of layer 302, 402, 502, 602 may be any adhesive suitable for attachment to a garment or a similar substrate, e.g., a pressure sensitive adhesive, a hot melt adhesive, etc. In some embodiments, the adhesive may be applied to the entire surface of layer 302, 402, 502, 602, or to predetermine thereon. In some embodiments, layer 302, 402, 502, 602 may be attached to the garment by any appropriate joining technique, such as by sewing, attachment through a hook and loop-type fastener, buttons, and the like.

In some embodiments, attachment layer 302, 402, 502, 602 may be configured to be detachably removed and re-attached, e.g., to enable relocating of telemedicine device 300, 400, 500, 600 to another location within the garment. In some embodiments, attachment layer 302, 402, 502, 602 may be configured for removal and re-attachment one or more times without significant deterioration in the securement of attachment layer 302, 402, 502, 602 to the garment. In some embodiments, at least attachment layer 302, 402, 502, 602 may be at least partially deformable to allow at least attachment layer 302, 402, 502, 602 to generally conform to the surface topology of the garment.

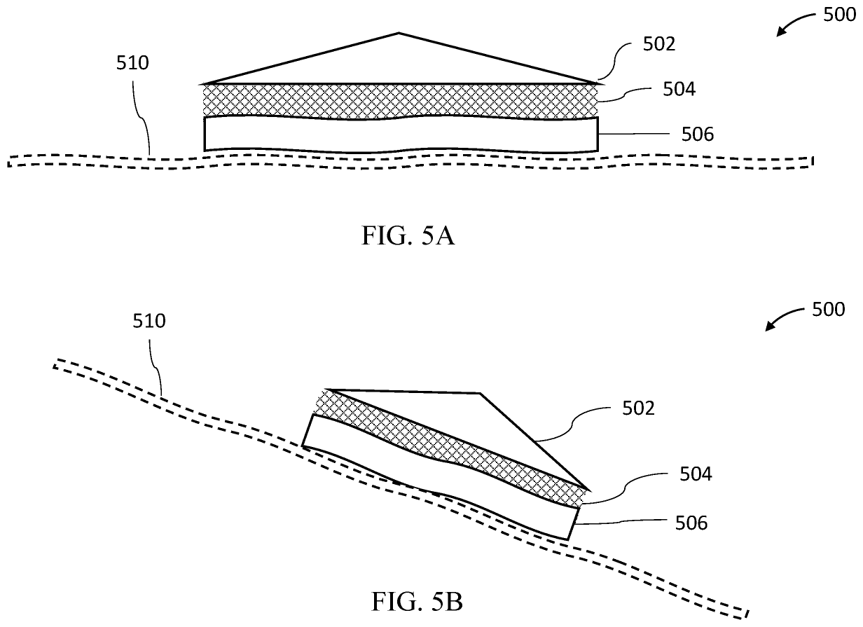

In some embodiments, layer 302, 402, 502, 602 may be shaped and dimensioned to increase a local force exerted with respect to each telemedicine device 300, 400, 500, 600 by a garment of the present disclosure, so as to enhance locally the amount of resilient force applied to each telemedicine device 300, 400, 500, 600. For example, as can be seen in FIGS. 4A-4B, which are side and perspective views, respectively, of a telemedicine device 400 of the present disclosure, attachment layer 402 may comprise a hemispherical or dome-like attachment layer. Similarly, as can be seen in FIGS. 5A-5B, which are side and perspective views, respectively, of a telemedicine device 500 of the present disclosure, attachment layer 502 may comprise a pyramidal or conical attachment layer. In other embodiments, an attachment layer of a telemedicine device 300, 400, 500, 600 of the present disclosure may define any three-dimensional shape generally tapering to a defined vertex point or a region from a base plane, e.g., resembling a conical, pyramidal, cylindrical, cuboidal, and/or any similar shape. In some embodiments, attachment layer may generally resemble a truncated 3D volumetric shape, e.g., a truncated conical, pyramidal, cylindrical, cuboidal, and/or any similar shape, culminating in a top and/or forward plane.

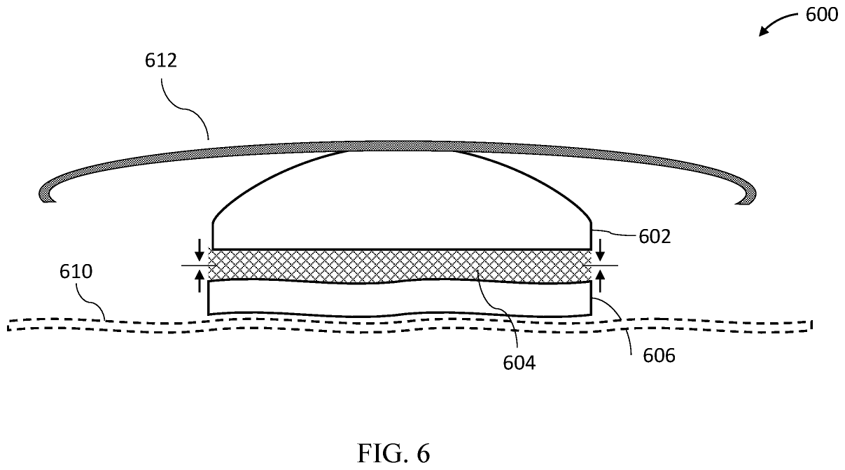

FIG. 6 shows a telemedicine device 600 of the present disclosure attached to a section of garment 612 worn by a subject. Telemedicine device 600 may comprise, e.g., an attachment layer 602, a resilient layer 604, and a proximate layer 606 in contact with skin 610 of the subject. Garment section 612 may exert a (perpendicular) force urging telemedicine device towards contact with skin 610, which tends to secure a positioning of telemedicine device 600 at a predetermined location on the subject's skin 610. Resilient layer 604 cooperates with the garment section 612 to exert additional force locally with respect to telemedicine device 600 so as to enhance locally the amount of resilient force applied to telemedicine device 600.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims, each of the terms “substantially,” “essentially,” and forms thereof, when describing a numerical value, means up to a 20% deviation (namely, ±20%) from that value. Similarly, when such a term describes a numerical range, it means up to a 20% broader range—10% over that explicit range and 10% below it).

In the description, any given numerical range should be considered to have specifically disclosed all the possible subranges as well as individual numerical values within that range, such that each such subrange and individual numerical value constitutes an embodiment of the present disclosure. This applies regardless of the breadth of the range. For example, description of a range of integers from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 4, and 6. Similarly, description of a range of fractions, for example from 0.6 to 1.1, should be considered to have specifically disclosed subranges such as from 0.6 to 0.9, from 0.7 to 1.1, from 0.9 to 1, from 0.8 to 0.9, from 0.6 to 1.1, from 1 to 1.1 etc., as well as individual numbers within that range, for example 0.7, 1, and 1.1.

The descriptions of the various embodiments of the present invention have been presented for purposes of illustration, but are not intended to be exhaustive or limited to the explicit descriptions. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise,” “include,” and “have,” as well as forms thereof, are not necessarily limited to members in a list with which the words may be associated.

Where there are inconsistencies between the description and any document incorporated by reference or otherwise relied upon, it is intended that the present description controls.

1. A wearable system comprising:
a body covering configured to cover a body part of a subject;
at least one device attached to a surface of said body covering at a predetermined location with respect to said body part, said at least one device comprising:
a skin-proximate layer configured to engage with a skin of said subject in a contact engagement at said predetermined location,
an attachment layer configured to attach to said surface of said body covering, and
a resilient layer disposed between said skin-proximate layer and said attachment layer,
wherein said body covering and said resilient layer are configured to cooperate to exert a combined force ensures long-term consistent positioning of said skin-proximate layer at said predetermined location.

2. The wearable system of claim 1, wherein said skin-proximate layer comprises at least one sensor configured to produce a signal representative of a physiological characteristic of said subject, wherein said at least one sensor is selected from one of the following categories of sensors: heart monitoring sensors, heart-rate monitoring sensors, electrocardiogram (ECG) sensors, body temperature sensors, breath-rate/respiration sensors, skin conductivity sensors, oxygen saturation sensors, blood perfusion sensors, blood pressure sensor, skin humidity sensors, electroencephalograph (EEG) sensors, muscle activity sensors, location sensors, and accelerometers.

3. (canceled)

4. The wearable system of claim 1, wherein said device comprises at least one therapy element configured to administer a therapy to said subject at said predetermined location, wherein said therapy element is selected from one of the following categories of therapy elements: injection delivery devices, medication delivery devices, signal delivery devices, defibrillators, or canisters configured to store a specified amount of a medication for application to said subject through an opening in said skin-proximate layer.

5. (canceled)

6. (canceled)

7. The wearable system of claim 1, wherein at least a portion of said body covering is formed with a predetermined amount of elasticity.

8. The wearable system of claim 1, wherein said device comprises a wireless transceiver configured to receive and transmit data to an external control module.

9. (canceled)

10. The wearable system of claim 1, wherein said attachment layer is configured to detachably attach to said surface of said body covering using one of: an adhesive, sewing, attachment through a hook and loop fastener, and buttons.

11. (canceled)

12. The wearable system of claim 1, wherein said device has a shape defining one of: a circle, an oval, a rectangle, a triangle, and a polygon, wherein a circle inscribing said device has a diameter of between 5-100 mm, wherein said device has a thickness at a thickest point thereof of between 0.25-25 mm, and wherein said resilient layer has a thickness of between 0.1-24.75 mm.

13. (canceled)

14. (canceled)

15. (canceled)

16. The wearable system of claim 1, wherein said skin-proximate layer comprises an electrode comprising at least one conductive segment configured to make electrical contact with said skin of said subject, and wherein said at least one conductive segment comprises one of the following options: two or more concentric circles extending inwardly from an outer circumference of said skin-proximate layer, or at least one continuous conductive spiral extending from an outer circumference of said skin-proximate layer to a center point of said skin-proximate layer.

17. (canceled)

18. The wearable system of claim 1, wherein said body covering is a garment selected from the group consisting of: shirt, pants, short pants, bodysuit, undergarment, undershirt, bra, headwear, headgear, footwear, armband, and leg band.

19. The wearable system of claim 1, wherein said body covering comprises an adhesive partial body covering or dressing configured to cover an area of a body.

20. A method comprising:
providing a wearable system comprising:
a body covering configured to cover a body part of a subject;
at least one device attached to a surface of said body covering at a predetermined location with respect to said body part, said at least one device comprising:
a skin-proximate layer configured to engage with a skin of said subject in a contact engagement at said predetermined location,
an attachment layer configured to attach to said surface of said body covering, and
a resilient layer disposed between said skin-proximate layer and said attachment layer,
wherein said body covering and said resilient layer are configured to cooperate to exert a combined force ensures long-term consistent positioning of said skin-proximate layer at said predetermined location; and
positioning said body covering about said body part so that said at least one device is located at said predetermined location and said skin-proximate layer engages with said skin of said subject in a contact engagement at said predetermined location.

21. The method of claim 20, wherein said skin-proximate layer comprises at least one sensor configured to produce a signal representative of a physiological characteristic of said subject, wherein said at least one sensor is selected from one of the following categories of sensors: heart monitoring sensors, heart-rate monitoring sensors, electrocardiogram (ECG) sensors, body temperature sensors, breath-rate/respiration sensors, skin conductivity sensors, oxygen saturation sensors, blood perfusion sensors, blood pressure sensor, skin humidity sensors, electroencephalograph (EEG) sensors, muscle activity sensors, location sensors, and accelerometers.

22. (canceled)

23. The method of claim 20, wherein said device comprises at least one therapy element configured to administer a therapy to said subject at said predetermined location, and wherein said method further comprises administering said therapy to said subject at said predetermined location, wherein said therapy element is selected from one of the following categories of therapy elements:
injection delivery devices, medication delivery devices, signal delivery devices, defibrillators, or canisters configured to store a specified amount of a medication for application to said subject through an opening in said skin-proximate layer.

24. (canceled)

25. (canceled)

26. The method of claim 20, wherein at least a portion of said body covering is formed with a predetermined amount of elasticity.

27. The method of claim 20, wherein said device comprises a wireless transceiver configured to receive and transmit data to an external control module.

28. (canceled)

29. The method of claim 20, wherein said attachment layer is configured to detachably attach to said surface of said body covering using one of: an adhesive, sewing, attachment through a hook and loop fastener, and buttons.

30. (canceled)

31. The method of claim 20, wherein said device has a shape defining one of: a circle, an oval, a rectangle, a triangle, and a polygon, wherein a circle inscribing said device has a diameter of between 5-100 mm, wherein said device has a thickness at a thickest point thereof of between 0.25-25 mm, and wherein said resilient layer has a thickness of between 0.1-24.75 mm.

32. (canceled)

33. (canceled)

34. (canceled)

35. The method of claim 20, wherein said skin-proximate layer comprises an electrode comprising at least one conductive segment configured to make electrical contact with said skin of said subject and wherein said at least one conductive segment comprises one of the following options: two or more concentric circles extending inwardly from an outer circumference of said skin-proximate layer, or at least one continuous conductive spiral extending from an outer circumference of said skin-proximate layer to a center point of said skin-proximate layer.

36. (canceled)

37. The method of claim 20, wherein said body covering is a garment selected from the group consisting of: shirt, pants, short pants, bodysuit, undergarment, undershirt, bra, headwear, headgear, footwear, armband, and leg band.

38. The method of claim 20, wherein said body covering comprises an adhesive partial body covering or dressing configured to cover an area of a body.

39.-48. (canceled)
